(19) United States
(12) Patent Application Publication
(10) Pub. No.: US 2025/0223652 A1
(43) Pub. Date: Jul. 10, 2025

(54) TIERED TESTING FOR HIGH RISK POPULATIONS

(71) Applicant: Harbinger Health, Inc., Cambridge, MA (US)

(72) Inventor: Anthony P. Shuber, Cambridge, MA (US)

(21) Appl. No.: 19/009,609

(22) Filed: Jan. 3, 2025

Related U.S. Application Data
(60) Provisional application No. 63/685,159, filed on Aug. 20, 2024.
(60) Provisional application No. 63/618,007, filed on Jan. 5, 2024.

(51) Int. Cl. C12Q 1/6886; C12Q 1/6806; C12Q 1/686; C12Q 1/6874; G16B 20/20; G16B 40/20; G16H 20/10; G16H 50/20

(52) U.S. Cl. CPC C12Q 1/6886; C12Q 1/6806; C12Q 1/686; C12Q 1/6874; G16B 20/20; G16B 40/20; G16H 20/10; G16H 50/20; C12Q 2600/154

(57) ABSTRACT

Disclosed herein are methods for detecting a false positive initial sample result in a subject initially identified as having, or at risk for, cancer. Such methods can be performed on a sample obtained from the subject while undergoing a colonoscopy. Thus, methods can be useful for confirming or contradicting the initial identification that the high risk subject is at risk for cancer. Altogether, such methods are valuable for improving precision of a cancer test.

### CROSS-REFERENCE TO RELATED APPLICATIONS

This application claims the benefit of and priority to U.S. Provisional Patent Application No. 63/685,159 filed Aug. 20, 2024, and to U.S. Provisional Patent Application No. 63/618,007 filed Jan. 5, 2024, the entire disclosure of each of which is hereby incorporated by reference in its entirety for all purposes.

### BACKGROUND

Commercial cancer tests are prevalent today and can return diagnostic results to patients in the comfort of their own home. However, such diagnostic methods are often inaccurate, returning false positive results that can cause patients to undertake unnecessary changes. There is a need for effective methods that identify patients who have received falsely positive diagnoses in a timely manner.

### SUMMARY

Disclosed herein are methods for detecting a false positive initial sample result in a subject initially identified as having, or at risk for, cancer. For example, the subject may have initially been identified as having, or at risk for, cancer by undergoing a prior analysis of the initial sample. Thus, disclosed methods are useful for confirming or contradicting the initial identification that the high risk subject is at risk for cancer. In various embodiments, methods involve further determining additional or orthogonal cancer information in relation to the prior analysis of the initial sample. Such methods are valuable for improving precision of a cancer test. In particular embodiments, methods are useful for detecting a false positive initial sample result in a subject initially identified as having, or at risk for, gastrointestinal (GI) cancer. Other forms of cancer can be readily adapted in accordance with the methods disclosed herein.

Disclosed herein is a method for detecting a true positive or a false positive initial sample result in a subject initially identified as having, or at risk for, cancer, the method comprising: analyzing methylation statuses of a plurality of genomic sites from target nucleic acids of a biological sample of the subject; determining whether the biological sample has a presence or absence of a cancer signal to confirm or contradict the initial identification that the high risk subject is at risk for cancer, wherein the absence of the cancer signal signifies that the biological sample does not have a presence of or is not at risk for a plurality of cancers, wherein at least one cancer of the plurality of cancers is a different cancer that differs from the cancer of the prior analysis; and classifying the initial sample as a true positive biological sample or a false positive biological sample responsive to the determination.

In various embodiments, the subject initially identified as at risk of cancer is undergoing or has previously undergone a colonoscopy. In various embodiments, the biological sample is obtained during a colonoscopy of the subject. In various embodiments, the cancer is any of esophageal cancer, stomach cancer, small intestine cancer, colorectal cancer, or rectal cancer. In various embodiments, the initial sample result was obtained using an initial cancer diagnostic test. In various embodiments, the cancer diagnostic test analyzed an initial blood or stool sample of the subject. In various embodiments, the initial blood or stool sample and the biological sample are different samples. In various embodiments, the initial cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample, In various embodiments, the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

In various embodiments, determining that the sample lacks the cancer signal comprises determining cancer information that is orthogonal to the information from the previous cancer diagnostic test. In various embodiments, the different cancer is one of acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the different cancer is one or more of liver or lung cancer. In various embodiments, the absence of the cancer signal signifies that the sample does not have or is not at risk for liver or lung cancer.

In various embodiments, the plurality of genomic sites comprise a plurality of CpG sites. In various embodiments, the plurality of CpG sites are located in one or more CpG islands or portions of one or more CpG islands shown in Tables 1-4. In various embodiments, the methylation statuses of the plurality of genomic sites are obtained by performing an assay, wherein the assay comprises performing one or more of: a. sequencing of target nucleic acids; b. hybrid capture; c. methylation-specific PCR; d. an assay that generates sequence information; and e. sequencing a clone library generated from a template immortalized library. In various embodiments, performing the assay comprises: obtaining converted cell free DNA (cfDNA); selectively amplifying target regions of the converted cfDNA; and sequencing amplicons comprising the amplified target regions to determine the methylation statuses of the plurality of genomic sites.

In various embodiments, the converted cfDNA comprises bisulfite converted cfDNA. In various embodiments, obtaining the converted cfDNA comprises performing bisulfite conversion of the target nucleic acids of the sample. In various embodiments, the sample from the high risk subject comprises any one of a blood sample, a stool sample, a urine sample, a mucous sample, or a saliva sample. In various embodiments, the sample from the high risk subject is a blood sample. In various embodiments, analyzing methylation statuses of the plurality of genomic sites from target nucleic acids of the sample comprises performing whole genome sequencing. In various embodiments, analyzing methylation statuses of the plurality of genomic sites from target nucleic acids of the sample comprises applying a trained machine learning model. In various embodiments, the target nucleic acids comprise cell free DNA (cfDNA). In various embodiments, classifying the initial sample as a true positive sample or a false positive sample responsive to the determination comprises classifying the initial sample as a false positive responsive to determining that the sample has an absence of the cancer signal.

In various embodiments, methods disclosed herein further comprise selecting a health regimen for the high risk subject that does not include a tumor therapeutic. In various embodiments, classifying the initial sample as a true positive sample or a false positive sample responsive to the determination comprises classifying the initial sample as a true positive responsive to determining that the sample has a presence of the cancer signal. In various embodiments, methods disclosed herein further comprise selecting a tumor therapeutic for administration to the high risk subject. In various embodiments, the method detects false positive samples at an accuracy of at least 80% sensitivity at 90% specificity.

Additionally disclosed herein is a method for improving precision of a cancer test, the method comprising: identifying a subject at risk for cancer due to an initial sample result from a cancer diagnostic test; analyzing methylation statuses of a plurality of genomic sites from target nucleic acids of a biological sample of the subject; determining whether the biological sample has a presence or absence of a cancer signal to confirm or contradict the initial identification that the high risk subject is at risk for cancer, wherein the absence of the cancer signal signifies that the biological sample does not have a presence of or is not at risk for a plurality of cancers, wherein at least one cancer of the plurality of cancers is a different cancer; and classifying the initial sample as a true positive biological sample or a false positive biological sample responsive to the determination.

In various embodiments, the subject is undergoing or has previously undergone a colonoscopy. In various embodiments, the biological sample is obtained during a colonoscopy of the subject. In various embodiments, the cancer is any of esophageal cancer, stomach cancer, small intestine cancer, colorectal cancer, or rectal cancer. In various embodiments, the cancer diagnostic test analyzed an initial blood or stool sample of the subject. In various embodiments, the initial blood or stool sample and the biological sample are different samples. In various embodiments, the initial cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample, In various embodiments, the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test. In various embodiments, determining that the sample lacks the cancer signal comprises determining cancer information that is orthogonal to the information from the previous cancer diagnostic test. In various embodiments, the different cancer is one of acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the different cancer is one or more of liver or lung cancer. In various embodiments, the absence of the cancer signal signifies that the sample does not have or is not at risk for liver or lung cancer. In various embodiments, the plurality of genomic sites comprise a plurality of CpG sites. In various embodiments, the plurality of CpG sites are located in one or more CpG islands or portions of one or more CpG islands shown in Tables 1-4.

In various embodiments, the methylation statuses of the plurality of genomic sites are obtained by performing an assay, wherein the assay comprises performing one or more of: a. sequencing of target nucleic acids; b. hybrid capture; c. methylation-specific PCR; d. an assay that generates sequence information; and e. sequencing a clone library generated from a template immortalized library. In various embodiments, performing the assay comprises: obtaining converted cell free DNA (cfDNA); selectively amplifying target regions of the converted cfDNA; and sequencing amplicons comprising the amplified target regions to determine the methylation statuses of the plurality of genomic sites. In various embodiments, the converted cfDNA comprises bisulfite converted cfDNA. In various embodiments, obtaining the converted cfDNA comprises performing bisulfite conversion of the target nucleic acids of the sample. In various embodiments, the sample from the high risk subject comprises any one of a blood sample, a stool sample, a urine sample, a mucous sample, or a saliva sample. In various embodiments, the sample from the high risk subject is a blood sample. In various embodiments, analyzing methylation statuses of the plurality of genomic sites from target nucleic acids of the sample comprises performing whole genome sequencing. In various embodiments, analyzing methylation statuses of the plurality of genomic sites from target nucleic acids of the sample comprises applying a trained machine learning model. In various embodiments, the target nucleic acids comprise cell free DNA (cfDNA). In various embodiments, classifying the initial sample as a true positive sample or a false positive sample responsive to the determination comprises classifying the initial sample as a false positive responsive to determining that the sample has an absence of the cancer signal. In various embodiments, methods disclosed herein further comprise selecting a health regimen for the high risk subject that does not include a tumor therapeutic. In various embodiments, classifying the initial sample as a true positive sample or a false positive sample responsive to the determination comprises classifying the initial sample as a true positive responsive to determining that the sample has a presence of the cancer signal. In various embodiments, methods disclosed herein further comprise selecting a tumor therapeutic for administration to the high risk subject. In various embodiments, the method detects false positive samples at an accuracy of at least 80% sensitivity at 90% specificity. In various embodiments, prior to the subject having been initially identified as having, or at risk for, cancer, monitoring the subject over one or more timepoints. In various embodiments, monitoring the subject over one or more timepoints comprises determining levels of a plurality of biomarkers across the one or more timepoints.

Additionally disclosed herein is a method for improving precision of one or more cancer tests, the method comprising: for each of one or more previously screened subjects of a patient population; analyzing methylation statuses of a plurality of genomic sites from target nucleic acids of a biological sample of the previously screened subject; determining whether the biological sample of the previously screened subject has a presence or absence of a cancer signal to confirm or contradict an initial identification using an initial sample obtained from the previously screened subject, wherein the absence of the cancer signal signifies that the biological sample does not have a presence of or is not at risk for a plurality of cancers; and classifying the initial sample as a true positive biological sample or a false positive biological sample responsive to the determination. In various embodiments, at least one cancer of the plurality of cancers is a different cancer that differs from the cancer for which the previously screened subject was initially identified as high risk. In various embodiments, the method achieves at least a 60% positive predictive value. In various embodiments, the method achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In various embodiments, each of the one or more previously screened subjects is a symptomatic patient exhibiting one or more symptoms of a cancer. In various embodiments, the initial identification using the initial sample obtained from the previously screened subject was determined using a cancer diagnostic test that analyzed an initial blood or stool sample of the previously screened subject. In various embodiments, the initial blood or stool sample and the biological sample are different samples. In various embodiments, the cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample, In various embodiments, the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

In various embodiments, prior to the previously screened subject having been initially identified as having, or at risk for, cancer, monitoring the subject over one or more timepoints. In various embodiments, monitoring the subject over one or more timepoints comprises determining levels of a plurality of biomarkers across the one or more timepoints.

Additionally disclosed is a method for improving precision of a cancer test for detecting cancer in subjects of a patient population, the method comprising: monitoring subjects of the patient population by screening the subjects using initial samples obtained from the subjects, wherein the screening comprises performing an initial cancer diagnostic test; identifying one or more subjects at risk for cancer based on results from the initial cancer diagnostic test; for each of the one or more subjects at risk for cancer: analyzing methylation statuses of a plurality of genomic sites from target nucleic acids of a biological sample of the subject; determining whether the biological sample has a presence or absence of a cancer signal to confirm or contradict the initial identification that the subject is at risk for cancer based on results from the initial cancer diagnostic test, wherein the absence of the cancer signal signifies that the biological sample does not have a presence of or is not at risk for a plurality of cancers; and classifying the results from the initial cancer diagnostic test as a true positive result or a false positive result responsive to the determination.

In various embodiments, at least one cancer of the plurality of cancers is a different cancer that differs from the cancer for which the subject was initially identified as at risk based on results from the initial cancer diagnostic test. In various embodiments, the method achieves at least a 60% positive predictive value. In various embodiments, the method achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In various embodiments, at least one of the one or more subjects at risk for cancer is a symptomatic patient exhibiting one or more symptoms of a cancer. In various embodiments, the initial cancer diagnostic test analyzed an initial blood or stool sample of the subject. In various embodiments, the initial blood or stool sample and the biological sample are different samples. In various embodiments, the initial cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample, In various embodiments, the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test. In various embodiments, monitoring subjects of the patient population comprises monitoring the subjects over one or more timepoints. In various embodiments, monitoring the subjects over one or more timepoints comprises determining levels of a plurality of biomarkers across the one or more timepoints.

### BRIEF DESCRIPTION OF THE DRAWINGS

These and other features, aspects, and advantages of the present invention will become better understood with regard to the following description and accompanying drawings. It is noted that wherever practicable, similar or like reference numbers may be used in the figures and may indicate similar or like functionality. For example, a letter after a reference numeral, such as “sample 115A,” indicates that the text refers specifically to the element having that particular reference numeral. A reference numeral in the text without a following letter, such as “sample 115,” refers to any or all of the elements in the figures bearing that reference numeral (e.g. “sample 115” in the text refers to reference numerals “sample 115A” and/or “sample 115B” in the figures).

FIG. 1 depicts an overall flow process for improving precision of a cancer test, in accordance with an embodiment.

FIG. 2A depicts an example conversion of nucleic acids, in accordance with an embodiment.

FIG. 2B shows the results of nitrite conversion on select nucleotides, in accordance with a second embodiment. Figure adapted from Li et al. (2022) Genome Biology 23:122.

FIG. 3A depicts example methylation information useful for improving precision of a cancer test, in accordance with an embodiment.

FIG. 3B shows an example flow process for determining whether an individual is at risk for a cancer, in accordance with an embodiment.

FIG. 4 shows an example flow diagram for improving precision of a cancer test, in accordance with a first embodiment.

FIG. 5 illustrates an example computer for implementing the entities shown in FIGS. 1, 2A-2B, 3A-3B, and 4.

### DETAILED DESCRIPTION

### Definitions

Terms used in the claims and specification are defined as set forth below unless otherwise specified.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “obtaining information” and “obtaining sequence information” encompasses obtaining information that is determined from at least one sample. Obtaining information (e.g., sequence information) encompasses obtaining a sample and processing the sample to experimentally determine the information (e.g., sequence information). The phrase also encompasses receiving the information, e.g., from a third party that has processed the sample to experimentally determine the information.

The phrase “target nucleic acids” refers to nucleic acids of an individual that contain at least signatures that may be informative for determining presence or absence of cancer. In various embodiments, target nucleic acids may be nucleic acids derived from a diseased cell that is associated with cancer. For example, target nucleic acids may be cell-free nucleic acids originating from cancer cells. Target nucleic acids can be any of DNA, cDNA, or RNA. In particular embodiments, target nucleic acids include DNA.

It must be noted that, as used in the specification, the singular forms “a,” “an” and “the” include plural referents unless the context clearly dictates otherwise.

### Overview

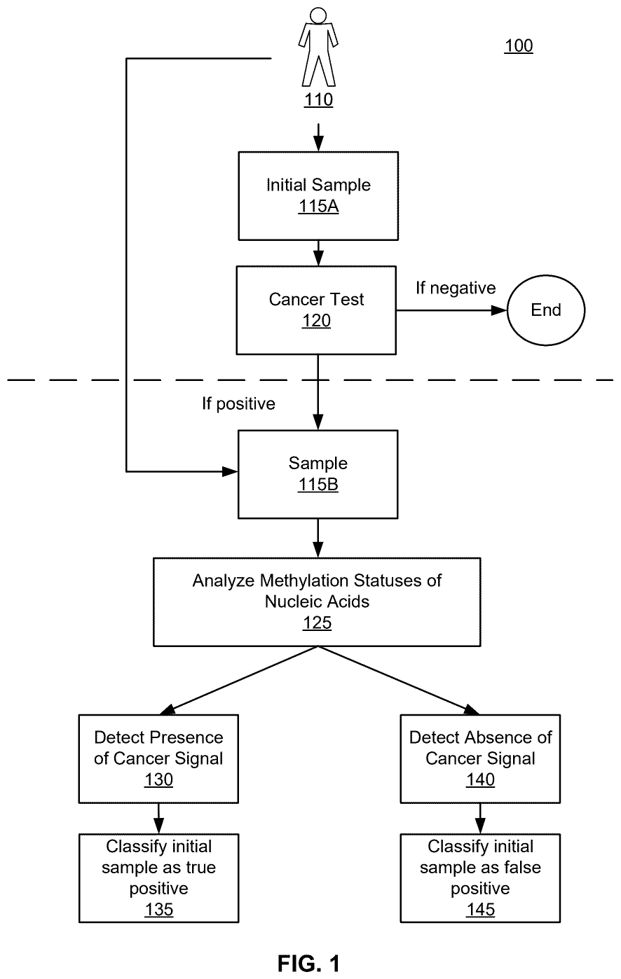

Disclosed herein are methods for detecting a true positive or a false positive initial sample result in a subject initially identified as having, or at risk for, cancer. Reference is made to FIG. 1, which depicts an overall flow process 100 of the disclosed methods, in accordance with an embodiment. For example, such methods are useful for detecting a true positive or a false positive initial sample and/or improving precision of a cancer test, such as a gastrointestinal (GI) cancer test.

FIG. 1 introduces a subject 110. Although FIG. 1 shows the flow process in relation to a single subject 110, in various embodiments, the flow process can be performed for more than a single subject 110 (e.g., for thousands, millions, tens of millions, or hundreds of millions of individuals).

Generally, the subject 110 may undergo an initial analysis that involves analyzing an initial sample 115A using a cancer test 120. As shown in FIG. 1, if the cancer test 120 returns a negative result (e.g., absence of cancer or absence of risk of cancer for the subject 110), then the process may end. Alternatively, if the cancer test 120 returns a positive result (e.g., presence of cancer or presence of risk of cancer for the subject 110) then a subsequent analysis can be performed to confirm or contradict the initial analysis. In embodiments where large numbers of subjects (e.g., hundreds, thousands, or even millions of subjects) undergo the cancer test 120, then the subsequent analysis need only be performed on a subset of the subjects who were deemed positive, referred to herein as high risk subjects and/or high risk population. Thus, only high risk subjects (e.g., subjects previously identified as having cancer or at risk of cancer) need undergo the subsequent analysis. If the subsequent analysis detects presence of a cancer signal (e.g., step 130), the subsequent analysis confirms the initial analysis by classifying the initial sample as a true positive biological sample at step 135. As another example, if the subsequent analysis detects absence of a cancer signal (e.g., step 140), the subsequent analysis contradicts the initial analysis by classifying the initial sample as a false positive biological sample at step 145.

As shown in FIG. 1, one or more samples (e.g., initial sample 115A and/or sample 115B) are obtained from the subject 110. In various embodiments, a sample is any of a blood sample, a stool sample, a urine sample, a mucous sample, or a saliva sample. In particular embodiments, each sample obtained from the subject 110 is a blood sample. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, the one or more samples can be obtained from the subject 110 by a reference lab.

In various embodiments, a plurality of samples are obtained from the subject 110 at a plurality of different points in time. For example, a sample (e.g., initial sample 115A) can be obtained at a first timepoint and at least a second sample (e.g., sample 115B) can be obtained from the subject 110 at a second timepoint. In various embodiments, the initial sample 115A and/or the sample 115B are each liquid biopsy samples. Obtaining a plurality of samples from the subject at a plurality of different points in time includes obtaining a number M of liquid biopsy samples, wherein M is one of: 2, 3, 4, . . . , N−1, N, wherein N is a positive integer.

In various embodiments, each sample (e.g., initial sample 115A and/or sample 115B) may include various biomarkers, examples of which include proteins, metabolites, and/or nucleic acids. In particular embodiments, the liquid biopsy sample includes cell-free DNA (cfDNA) fragments. In particular embodiments, the cfDNA fragments include genomic sequences corresponding to CpG islands for which methylation states are informative of the cancer.

Although not explicitly shown in FIG. 1, one or more assays can be performed on the initial sample 115A and/or the sample 115B. In various embodiments, initial sample 115A and/or sample 115B may be processed to extract target nucleic acids. In various embodiments, samples can undergo cellular disruption methods (e.g., to obtain genomic DNA) involving chemical methods or mechanical methods. Example chemical methods include osmotic shock, enzymatic digestion, detergents, or alkali treatment. Example mechanical methods include homogenization, ultrasonication or cavitation, pressure cell, or ball mill. In various embodiments, samples can undergo removal of membrane lipids or proteins or nucleic acid purification. Example chemical methods for removing membrane lipids or proteins and methods for nucleic acid purification include guanidine thiocyanate (GuSCN)-phenol-chloroform extraction, alkaline extraction, cesium chloride gradient centrifugation with ethidium bromide, Chelex® extraction, or cetyltrimethylammonium bromide extraction. Example physical methods for removing membrane lipids or proteins and methods for nucleic acid purification include solid-phase extraction methods using any of silica matrices, glass particles, diatomaceous earth, magnetic beads, anion exchange material, or cellulose matrix. Further details of nucleic acid extraction methods are described in Ali et al, Current Nucleic Acid Extraction Methods and Their Implications to Point-of-Care Diagnostics, Biomed Res. Int. 2017; 2017:9306564, which is hereby incorporated by reference in its entirety.

Referring first to the cancer test 120, in various embodiments, the cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample. In particular embodiments, the cancer test 120 is a gastrointestinal (GI) cancer test that detects risk of a presence or absence of GI cancer. In various embodiments, the cancer diagnostic test is a commercially available cancer diagnostic test. Examples of a cancer diagnostic test include any of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test. Generally, the cancer test 120 may determine that the subject 110 is negative for cancer (e.g., absence of cancer or lack of risk for cancer) or is positive for cancer (e.g., presence of cancer or presence of risk for cancer).

In various embodiments, following a positive diagnosis for cancer (e.g., due to the cancer test 120), the subject 110 can be deemed eligible for one or more additional procedures. For example, the subject 110 can be deemed eligible for a colonoscopy and/or a tissue biopsy. In various embodiments, following a positive diagnosis for cancer (e.g., due to the cancer test 120), the subject 110 can undergo one or more additional procedures. For example, the subject 110 can undergo a colonoscopy and/or a tissue biopsy.

As shown in FIG. 1, a sample 115B may be obtained from the subject 110 at a subsequent time if the cancer test 120 determines that the subject 110 is positive for a cancer. In various embodiments, the sample 115B may be obtained from the subject 110 e.g., by a medical professional during a visit at a reference laboratory or at a doctor's office. In various embodiments, the sample 115B may be obtained from the subject 110 during a visit at a doctor's office. In particular embodiments, the sample 115B can be obtained from the subject 110 during or close to when the subject 110 is undergoing one or more additional procedures. For example, the sample 115B can be obtained from the subject 110 on the same day that the subject 110 is undergoing a colonoscopy and/or a tissue biopsy. As another example, the sample 115B can be obtained from the subject 110 while the subject 110 is undergoing a colonoscopy and/or a tissue biopsy (e.g., sample 115B can be obtained from the subject 110 while the subject 110 is under anesthesia for the colonoscopy and/or tissue biopsy). In various embodiments, the sample 115B may be obtained from the subject 110 after the subject 110 has undergone the colonoscopy.

In various embodiments, after obtaining the sample 115B, methods involve performing one or more assays on the sample 115B e.g., to generate sequence information for a plurality of genomic sites of nucleic acids in the sample 115B. In various embodiments, the sequence information corresponds to a limited number of genomic sites that are sufficient for identifying whether the sample 115B includes a presence or absence of a cancer signal. In particular embodiments, the sequence information for a plurality of genomic sites includes methylation information, such as methylation statuses for the plurality of genomic sites. In various embodiments, the plurality of genomic sites include a plurality of CpG islands (CGIs) whose differential methylation status may be indicative of risk for the cancer. Further details of exemplary assays are described herein.

Step 125 involves analyzing methylation statuses of the nucleic acids of the sample 115B to determine whether the sample 115B has a presence or absence of a cancer signal. Thus, whether the sample 115B has a presence or absence of a cancer signal can be used to confirm or contradict the initial identification (e.g., from the cancer test 120) that the high risk subject is at risk for cancer.

In various embodiments, step 125 involves deploying a trained machine learning model to analyze methylation statuses of nucleic acids 125. Methods for training and deploying a trained machine learning model to analyze methylation statuses is described in WO20230147567, which is incorporated by reference in its entirety. Further details of analyzing methylation statuses of target nucleic acids is described herein.

Generally, step 125 either determines that the sample 115B includes a presence of a cancer signal (e.g., thus methods involve step 130 of detecting presence of a cancer signal in the sample 115B) or determines that the sample 115B does not include a presence of a cancer signal (e.g., thus methods involve step 140 of detecting absence of a cancer signal in the sample 115B). Thus, if presence of the cancer signal is detected at step 130, then the initial sample 115A is classified as a true positive sample at step 135. Here, the process confirms the initial identification that the subject 110 is a high risk subject at risk for cancer. In such embodiments, methods can involve selecting a treatment regimen that includes a tumor therapeutic for administration to the subject 110 to treat the cancer. In contrast, if absence of the cancer signal is detected at step 140, then the initial sample 115A is classified as a false positive sample at step 145. Here, the process contradicts the initial identification that the subject 110 is a high risk subject at risk for cancer. In such embodiments, methods can involve selecting a health regimen that does not involve a tumor therapeutic. For example, a health regimen that does not involve a tumor therapeutic can include lifestyle changes (e.g., sleep habits and exercise habits).

Generally, step 125 of analyzing methylation statuses of nucleic acids is able to effectively distinguish samples with a cancer signal and samples without a cancer signal. In particular embodiments, step 125 is able to distinguish samples with a cancer signal and samples without a cancer signal at an improved performance metric comparison to the cancer test 120. In various embodiments, step 125 detects false positive samples at an accuracy of at least 80% sensitivity at 90% specificity. Thus, methods involving step 125 are useful for detecting a true positive or a false positive initial sample, thereby confirming or contradicting the initial identification that the high risk subject is at risk for cancer. Altogether, this improves precision of a cancer test that outperforms the cancer test 120 by itself.

### Assays

Methods disclosed herein involve performing an assay to generate methylation information (e.g., methylation statuses of a plurality of genomic sites). Assays described in this section can be deployed to analyze either of the initial sample 115A or the sample 115B shown in FIG. 1. In various embodiments, performing an assay can include performing one or more of quantitative PCR (qPCR) or digital PCR (dPCR). In various embodiments, performing an assay comprises performing one or more of sequencing of target nucleic acids; hybrid capture; methylation-specific PCR; an assay that generates sequence information; and sequencing a clone library generated from a template immortalized library.

Generally, methylation information refers to methylation statuses for a plurality of genomic sites. In various embodiments, the plurality of genomic sites are previously identified and selected. For example, the plurality of genomic sites may be one or more CpG sites whose differential methylation are informative for determining whether an individual is at risk for a cancer. A CpG site is portion of a genome that has cytosine and guanine separated by only one phosphate group and is often denoted as “5′—C-phosphate-G—3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.” The methylation information can be analyzed to generate a prediction for a subject (e.g., whether a sample obtained from the subject includes a presence or absence of a cancer signal).

In various embodiments, performing an assay to generate sequence information for a plurality of genomic sites includes the steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences (e.g., via sequencing or via quantitative methods such as an ELISA, quantitative PCR, or DNA or RNA-based assay). In various embodiments, performing an assay to generate sequence information for a plurality of genomic sites involves a subset of the previously mentioned steps. For example, enriching the processed nucleic acids can be omitted. Therefore, performing an assay may include processing nucleic acids of a sample, amplifying the pre-selected genomic sequences, and quantifying the amplicons including the genomic sequences.

In various embodiments, the sequence information includes methylation statuses of two or more, three or more, four or more, five or more, six or more, seven or more, eight or more, nine or more, or ten or more genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In various embodiments, the plurality of genomic sites include a plurality of CpG islands (CGIs) whose differential methylation status may be indicative of a cancer.

A methylated nucleic acid is a nucleic acid having a modification in which a hydrogen atom on the pyrimidine ring of a cytosine base is converted to a methyl group, forming 5-methylcytosine. Methylation can occur at dinucleotides of cytosine and guanine referred to herein as “CpG sites”, which can be a target for enrichment. Methylation of cytosine can occur in cytosines in other sequence contexts, for example, 5′—CHG—3′ and 5′—CHH—3′, where H is adenine, cytosine or thymine. Cytosine methylation can also be in the form of 5-hydroxymethylcytosine. Methylation of DNA can include methylation of non-cytosine nucleotides, such as N⁶-methyladenine (6 mA). Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

In certain embodiments, the nucleic acid comprises a CpG site (i.e., cytosine and guanine separated by only one phosphate group). A CpG site is portion of a genome that has cytosine and guanine separated by only one phosphate group and is often denoted as “5′—C-phosphate-G—3′”, or “CpG” for short. In certain embodiments, the nucleic acid comprises a CpG island (also referred to as a “CG islands” or “CGI”) or a portion thereof, which is the target for enrichment. Because certain CGIs and certain features of certain CGIs in tumor cells tend to be different from the same CGIs or features of the CGIs in healthy cells, detection of such CGIs can be informative of a cancer. In certain embodiments, the CGI is a “cancer informative CGIs”, which is defined and described in more detail below. In certain embodiments, the CpG is an “informative CpG”, e.g., a “cancer informative CGI”. Such CGIs may have methylation patterns in tumor cells that are different from the methylation patterns in healthy cells. Accordingly, detection of a cancer informative CGI can be informative regarding a subject's risk of developing cancer or can be indicative that the subject has cancer. Exemplary cancer informative CGIs, which can be target sequences as described herein, are identified in, e.g., Table 1 of U.S. Patent Publication 2020/0109456A1, Tables 2 and 3 of WO2022/133315, and Tables 1-4 provided herein. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

In certain aspects, the nucleic acids have been treated to convert one or more unmethylated nucleotides (e.g., cytosines) to another nucleotide (a “converted nucleotide”, as used herein, such as a uracil), for example, prior to amplification. Example conversions include bisulfite conversion, enzymatic conversion, or nitrite conversion, further details of which are described herein. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5mC)) is converted to a thymine, which pairs with adenine. In certain embodiments, methylated cytosines are protected from conversion (e.g., deamination) during the conversion step.

In various embodiments, nucleic acids undergo a bisulfite conversion. Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

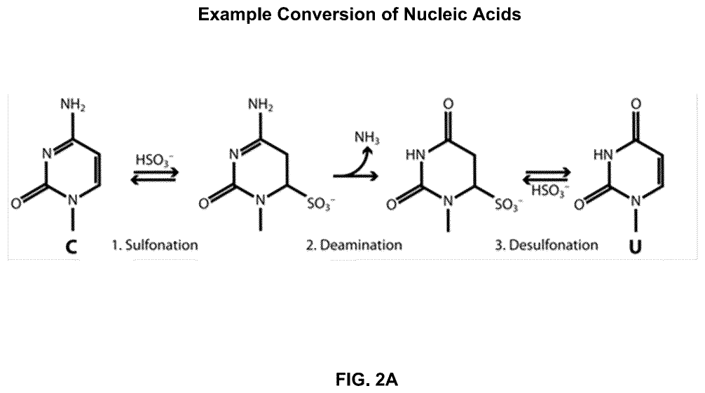

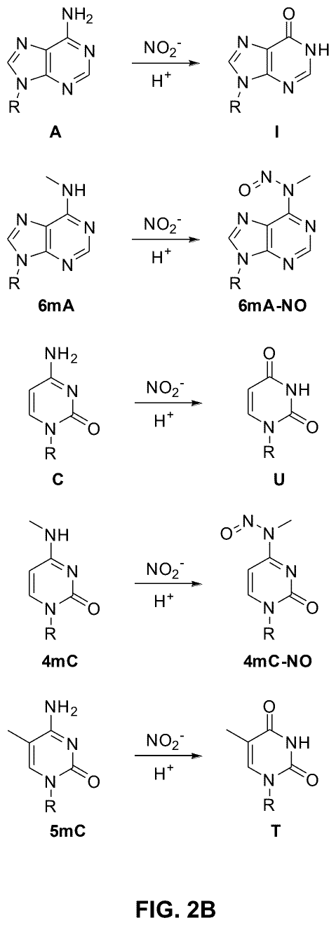

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. As shown in FIG. 2A, the steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. (See Clark et al., Nucleic Acids Res., 22(15):2990-7 (1994).) If the cytosine is methylated it will remain a methylated cytosine. If the cytosine is unmethylated it will be converted to uracil. When the modified strand is copied, for example, through extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

In various embodiments, nucleic acids undergo an enzymatic conversion. In certain embodiments, the enzymatic treatment with a cytidine deaminase enzyme is used to convert cytosine to uracil. Enzymatic conversion can include an oxidation step, in which Tet methylcytosine dioxygenase 2 (TET2) catalyzes the oxidation of 5 mC to 5 hmC to protect methylated cytosines from conversion by subsequent exposure to a cytidine deaminase. Other protection steps known in the art can be used in addition to or in place of oxidation by TET2. After the oxidation step, the nucleic acid is treated with the cytidine deaminase to convert one or more unmethylated cytosines to uracils. As with bisulfite conversion, when the modified strand is copied, a G will be incorporated in the interrogation position (opposite the C being interrogated) if the C was methylated and an A will be incorporated in the interrogation position if the C was unmethylated. When the double stranded extension product is amplified those Cs that were converted to Us and resulted in incorporation of A in the extended primer will be replaced by Ts during amplification. Those Cs that were not modified and resulted in the incorporation of G will remain as C.

In certain embodiments the cytidine deaminase may be APOBEC. In certain embodiments, the cytidine deaminase includes activation induced cytidine deaminase (AID) and apolipoprotein B mRNA editing enzymes, catalytic polypeptide-like (APOBEC). In certain embodiments, the APOBEC enzyme is selected from the human APOBEC family consisting of: APOBEC-1 (Apo1), APOBEC-2 (Apo2), AID, APOBEC-3A, -3B, -3C, -3DE, -3F, -3G, -3H and APOBEC-4 (Apo4). In certain embodiments, the APOBEC enzyme is APOBEC-seq.

In certain embodiments, nitrite treatment is used to deaminate adenine and cytosine. As shown in FIG. 2B, deamination of an A results in conversion to an inosine (I), which is read by a polymerase as a G, whereas deamination of a methylated A (N⁶-methyladenine (6mA)) results in a nitrosylated 6mA (6mA-NO), which causes the base to be read by a polymerase as an A. Deamination of a C results in conversion to a uracil, which is read by a polymerase as a T, whereas deamination of a N4-methylcytosine (4mC) to 4mC-NO or a 5-methylcytosine (5mC) to a T causes the base to be read by a polymerase as a C or a T, respectively. For 5mC bases, the C to T ratio at the 5mC position is about 40% higher than other cytosine positions, allowing 5mC to be differentiated from C. (Sec, Li et al. (2022) Genome Biology 23:122.)

In various embodiments, performing the assay includes enriching for specific genomic sequences, such as genomic sequences of pre-selected CGIs. In various embodiments, enrichment of pre-selected CGIs can be accomplished via hybrid capture. Examples of such hybrid capture probe sets include the KAPA HyperPrep Kit and SeqCAP Epi Enrichment System from Roche Diagnostics (Pleasanton, CA). For example, hybrid capture probe sets can be designed to target (e.g., hybridize with) selected genomic sequences, thereby capturing and enriching the selected genomic sequences.

In various embodiments, performing the assay includes a step of nucleic acid amplification. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

Examples of such assays include, but are not limited to performing PCR assays, Real-time PCR assays, Quantitative real-time PCR (qPCR) assays, digital PCR (dPCR), Allele-specific PCR assays, Reverse-transcription PCR assays and reporter assays. For example, given the processed nucleic acids (e.g., bisulfite converted nucleic acids) that are enriched for pre-selected genomic sequences, a PCR assay is performed to amplify the pre-selected genomic sequences to generate amplicons. Here, PCR primers are added to initiate the amplification. In various embodiments, the PCR primers are whole genome primers that enable whole genome amplification. In various embodiments, the PCR primers are gene-specific primers that result in amplification of sequences of specific genes. In various embodiments, the PCR primers are allele-specific primers. For example, allele specific primers can target a genomic sequence corresponding to a pre-selected CGI, such that performing nucleic acid amplification results in amplification of the genomic sequence of the pre-selected CGI.

In various embodiments, performing the assay includes quantifying the nucleic acids including the pre-selected genomic sequences (e.g., informative CGIs). In some embodiments, quantifying the nucleic acids to generate sequence information comprises performing an enzyme-linked immunosorbent assay (ELISA). In some embodiments, quantifying the nucleic acids to generate sequence information comprises performing quantitative PCR (qPCR) or digital PCR (dPCR). Therefore, the number of methylated, unmethylated, or partially methylated pre-selected genomic sequences can be quantified.

In various embodiments, quantifying the nucleic acids comprises sequencing the nucleic acids including the pre-selected genomic sequences. Thus, the sequenced reads can be aligned to a reference library and methylation sequence information including methylation statuses of the informative CGIs can be determined. Therefore, the number of methylated, unmethylated, or partially methylated pre-selected genomic sequences can be quantified via the sequenced reads.

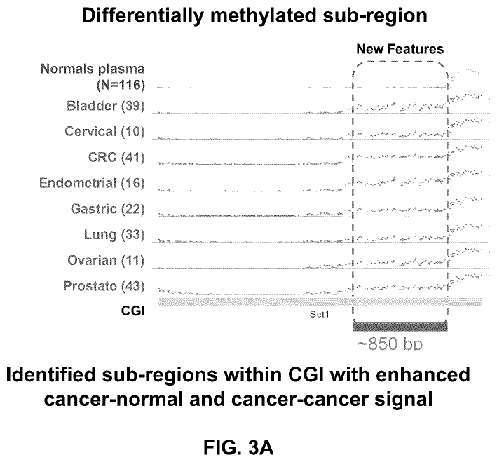

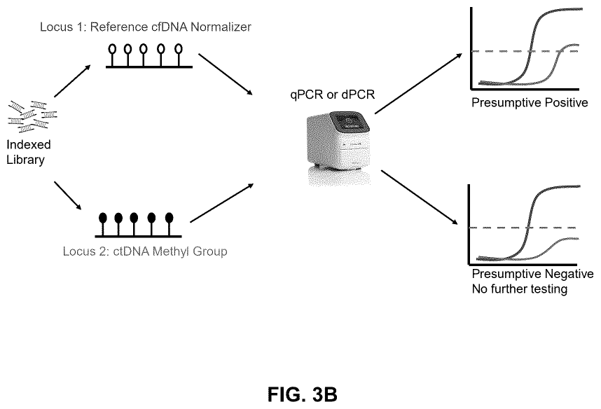

Reference is made to FIG. 3A, which depicts example methylation information useful for determining whether an individual is at risk for a cancer, in accordance with an embodiment. Specifically, FIG. 3A shows that across various types of cancers (e.g., bladder, cervical, colorectal, endometrial, gastric, lung, ovarian, and prostate cancers), sub-regions within a particular CGI can exhibit differential methylation in comparison to normal plasma. Thus, FIG. 3A depicts an example cancer informative CGI such that performing the assay results in the generation of methylation sequence information corresponding to the cancer informative CGI.

FIG. 3B shows an example flow process for determining whether an individual is at risk for a cancer, in accordance with an embodiment. Here, specific genomic regions of an indexed library of nucleic acids (e.g., DNA) are targeted. For example, locus I can refer to a reference genomic location. Here, a reference genomic location serves as a control. For example, the reference genomic location is not differentially methylated in healthy individuals in comparison to individuals with the cancer. Locus 2 can refer to a pre-selected genomic location, such as a pre-selected informative CGI.

Performing the assay further includes performing nucleic acid amplification (e.g., PCR) to generate methylation information. In various embodiments, nucleic acid amplification includes either qPCR or dPCR. This quantifies the number of methylated, unmethylated, or partially methylated sequences at locus 1 (reference) and at locus 2. In various embodiments, performing the assay includes performing an ELISA to quantify the number of methylated, unmethylated, or partially methylated sequences at locus 1 (reference) and at locus 2.

### Methods for Analyzing Methylation Statuses of Target Nucleic Acids

The description in this section pertains to at least step 125 of analyzing methylation statuses of nucleic acids (as shown in FIG. 1). Generally, the analysis is performed on methylation information generated from an assay having been performed on nucleic acids of a sample e.g., sample 115B shown in FIG. 1. In various embodiments, the analysis is performed to determine whether the sample obtained from the subject contains a cancer signal.

In various embodiments, the analysis is performed using a system comprising a computer storage and a processing system. The analysis can involve the implementation of trained machine learning models, details of which are described in further detail herein. For example, the computer storage can store sequence information corresponding to a processed sample, the processed sample including cell-free DNA fragments originating from a liquid biopsy of an individual and having been processed to enrich for cancer informative CGIs, the sequencer information comprising, for each sequenced cell-free DNA fragment corresponding to the cancer informative CGIs, a respective position on the genome for the cell-free DNA fragment and methylation information for the cell-free DNA fragment.

In various embodiments, the analysis involves analyzing a plurality of CGIs, or portions thereof. For example, the analysis involves analyzing methylation statuses of a plurality of CGIs, or portions thereof. Cancer informative CGI can be a “CGI identifier” or reference number to allow referencing CGIs during data processing by their respective unique CGI identifiers. The accompanying tables (e.g., Tables 1-4) lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In various embodiments, the analysis involves analyzing all of the CGIs in any one of Tables 1, 2, 3, or 4. In various embodiments, the analysis involves analyzing at least 10% of the CGIs in Table 1. In various embodiments, the analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 1. In various embodiments, the analysis involves analyzing at least 10% of the CGIs in Table 2. In various embodiments, the analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 2. In various embodiments, the analysis involves analyzing at least 10% of the CGIs in Table 3. In various embodiments, the analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 3. In various embodiments, the analysis involves analyzing at least 10% of the CGIs in Table 4. In various embodiments, the analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 4. In various embodiments, the analysis involves analyzing at least 10% of the CGIs in Tables 2 and 3. In various embodiments, the analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Tables 2 and 3.

In various embodiments, the analysis involves analyzing at least 100 CGIs (e.g., CGIs as shown in any of Tables 1-4). In various embodiments, the analysis involves analyzing at least 100 CGIs, at least 150 CGIs, at least 200 CGIs, at least 300 CGIs, at least 400 CGIs, at least 500 CGIs, at least 600 CGIs, at least 700 CGIs, at least 800 CGIs, at least 900 CGIs, at least 1000 CGIs, at least 1500 CGIs, at least 2000 CGIs, at least 2500 CGIs, at least 3000 CGIs, at least 3500 CGIs, at least 4000 CGIs, at least 4500 CGIs, at least 5000 CGIs, at least 5500 CGIs, or at least 6000 CGIs (e.g., CGIs as shown in any of Tables 1-4). In particular embodiments, performing the screen involves analyzing at least 500 CGIs. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

In various embodiments, the analysis achieves at least 60% sensitivity in detecting presence of a cancer signal. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the analysis achieves at least 85% sensitivity. In particular embodiments, the analysis achieves at least 86% sensitivity. In particular embodiments, the analysis achieves at least 87% sensitivity. In particular embodiments, the analysis achieves at least 88% sensitivity. In particular embodiments, the analysis achieves at least 89% sensitivity. In particular embodiments, the analysis achieves at least 90% sensitivity.

In various embodiments, the analysis achieves at least 60% specificity in excluding individuals without a cancer signal. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the analysis achieves at least 90% specificity. In particular embodiments, the analysis achieves at least 91% specificity. In particular embodiments, the analysis achieves at least 92% specificity. In particular embodiments, the analysis achieves at least 93% specificity. In particular embodiments, the analysis achieves at least 94% specificity. In particular embodiments, the analysis achieves at least 95% specificity.

In various embodiments, the analysis achieves at least 60% positive predictive value. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In particular embodiments, the analysis achieves at least 80% positive predictive value. In particular embodiments, the analysis achieves at least 81% positive predictive value. In particular embodiments, the analysis achieves at least 82% positive predictive value. In particular embodiments, the analysis achieves at least 83% positive predictive value. In particular embodiments, the analysis achieves at least 84% positive predictive value. In particular embodiments, the analysis achieves at least 85% positive predictive value.

In various embodiments, the analysis achieves at least 60% negative predictive value. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the analysis achieves at least 90% negative predictive value. In particular embodiments, the analysis achieves at least 91% negative predictive value. In particular embodiments, the analysis achieves at least 92% negative predictive value. In particular embodiments, the analysis achieves at least 93% negative predictive value. In particular embodiments, the analysis achieves at least 94% negative predictive value. In particular embodiments, the analysis achieves at least 95% negative predictive value. In particular embodiments, the analysis achieves at least 96% negative predictive value. In particular embodiments, the analysis achieves at least 97% negative predictive value. In particular embodiments, the analysis achieves at least 98% negative predictive value. In particular embodiments, the analysis achieves at least 99% negative predictive value.

### Machine Learning Models for Analyzing Sequence Information

In various embodiments, trained machine learning models can be deployed to analyze methylation statuses to determine whether a biological sample has a presence or absence of a cancer signal. In various embodiments, the trained machine learning models analyze differential methylation of the plurality of genomic sites to output predictions. Example methods for deploying a trained machine learning model to analyze methylation statuses is described in WO20230147567, which is incorporated by reference in its entirety.

In various embodiments, a trained machine learning model is deployed as part of the gastrointestinal cancer test (e.g., gastrointestinal test 120 as shown in FIG. 1). Thus, the trained machine learning model can analyze sequence information generated via an assay performed on the initial sample. In various embodiments, a trained machine learning model is deployed as part of step 125 analysis (e.g., step 125 involving analysis of methylation statuses of nucleic acids shown in FIG. 1). Therefore, the trained machine learning model can analyze sequence information including methylation statuses for a plurality of genomic sites, such as a plurality of CpG sites disclosed herein.

In various embodiments, a machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks).

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In particular embodiments, trained machine learning models analyze methylation statuses of a plurality of genomic sites to generate predictions. The methylation statuses can correspond to a set of cancer informative CpG islands (CGIs), wherein the cancer informative CGIs are selected from a group consisting of a ranked set of candidate CGIs. In various embodiments, a machine learning model analyzes methylation statuses for at least 50 CGIs, or portions thereof. In various embodiments, a machine learning model analyzes methylation statuses for at least 100 CGIs, or portions thereof. In various embodiments, a machine learning model analyzes methylation statuses for at least 150 CGIs, or portions thereof. In various embodiments, a machine learning model analyzes methylation statuses for at least 200 CGIs, or portions thereof. In various embodiments, a machine learning model analyzes methylation statuses for at least 250 CGIs, or portions thereof. In various embodiments, a machine learning model analyzes methylation statuses for at least 300 CGIs, or portions thereof. In various embodiments, a machine learning model analyzes methylation statuses for at least 400 CGIs, or portions thereof. In various embodiments, a machine learning model analyzes methylation statuses for at least 500 CGIs, or portions thereof. In various embodiments, a machine learning model analyzes methylation statuses for at least 600 CGIs, or portions thereof. In various embodiments, a machine learning model analyzes methylation statuses for at least 700 CGIs, or portions thereof. In various embodiments, a machine learning model analyzes methylation statuses for at least 800 CGIs, or portions thereof. In various embodiments, a machine learning model analyzes methylation statuses for at least 900 CGIs, or portions thereof. In various embodiments, a machine learning model analyzes methylation statuses for at least 1000 CGIs, or portions thereof. In various embodiments, a machine learning model analyzes methylation statuses for at least 2500 CGIs, or portions thereof. In various embodiments, a machine learning model analyzes methylation statuses for at least 5000 CGIs, or portions thereof. In various embodiments, a machine learning model analyzes methylation statuses for at least 7500 CGIs, or portions thereof. In various embodiments, a machine learning model analyzes methylation statuses for at least 10000 CGIs, or portions thereof. In various embodiments, a machine learning model analyzes methylation statuses for at least 15000 CGIs, or portions thereof. In various embodiments, a machine learning model analyzes methylation statuses for at least 20000 CGIs, or portions thereof. In various embodiments, a machine learning model analyzes methylation statuses for at least 25000 CGIs, or portions thereof. Such example CGIs, or portions thereof, are disclosed in the accompanying tables (e.g., Tables 1-4) which list, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety.

In various embodiments, a machine learning model analyzes methylation statuses for CGIs across the whole genome. For example, a machine learning model may be implemented to analyze sequencing data generated from whole genome sequencing (e.g., whole genome bisulfite sequencing).

### Monitoring and Pre-Screening Subjects of a Patient Population

In various embodiments, methods disclosed herein involve performing a multipart method using the multiple-tiered process (e.g., as disclosed in FIG. 1, the multiple-tiered process which includes a step 120 of a cancer test and a step 125 of analyzing methylation statuses of nucleic acids). In various embodiments, prior to performing the multiple-tiered process for a subject (e.g., prior to obtaining the initial sample 115A and performing a cancer test 120), the subject is monitored over time to e.g., determine when to perform the multiple-tiered process. In various embodiments, the subject is a healthy individual of a patient population and therefore, the subjects of the patient population can be monitored over time to e.g., determine when to perform the multiple-tiered process for certain subjects of the patient population. Thus, this ensures that not all subjects undergo the multiple-tiered process until a certain threshold is met. Here, the subjects of the patient population can be pre-screened over time to determine when to perform the multiple-tiered process for the subjects.

In various embodiments, the steps of obtaining the initial sample 115A and performing the cancer test 120, as shown in FIG. 1, refer to the monitoring and pre-screening steps. For example, an initial sample 115A can be obtained from the subject 110 (e.g., a subject from a patient population) and a cancer test 120 is performed as the subject 110 for monitoring and pre-screening purposes. In some scenarios, the analysis of the cancer test 120 reveals that the subject 110 is not at risk for cancer and thus, the subject 110 need not undergo additional testing. Thus, the subject 110 can continue to be monitored and pre-screened e.g., at a later time point. Thus, at the later time point, a new initial sample 115A and the cancer test 120 can be performed to determine whether the subject 110 is to undergo additional testing. If the additional testing is warranted, an additional sample 115B can be obtained from the subject 110 and step 125 (e.g., analyzing methylation statuses of nucleic acids) can be performed using the additional sample 115B. Thus, this ensures that not all subjects undergo the subsequent analysis (e.g., steps 115B and 125) until a certain threshold is met. Instead, certain subjects that have been monitored and pre-screened over time (e.g., through steps 115A and 120) are identified at particular time points for performing the subsequent analysis (e.g., steps 115B and 125).

In various embodiments, subjects of the patient population are monitored beginning at a threshold age. For example, subjects begin monitoring at a threshold age of 18 years old, 19 years old, 20 years old, 21 years old, 22 years old, 23 years old, 24 years old, 25 years old, 26 years old, 27 years old, 28 years old, 29 years old, 30 years old, 31 years old, 32 years old, 33 years old, 34 years old, 35 years old, 36 years old, 37 years old, 38 years old, 39 years old, 40 years old, 41 years old, 42 years old, 43 years old, 44 years old, 45 years old, 46 years old, 47 years old, 48 years old, 49 years old, or 50 years old. In particular embodiments, subjects begin monitoring at a threshold age of 18 years old. In particular embodiments, subjects begin monitoring at a threshold age of 20 years old. In particular embodiments, subjects begin monitoring at a threshold age of 21 years old.

In various embodiments, during monitoring, subjects of the patient population are tested for a plurality of biomarkers. For example, during monitoring for a subject, one or more samples can be obtained from the subject and tested for the plurality of biomarkers, where levels of the plurality of biomarkers or a change in the levels of the plurality of biomarkers can be informative for risk of cancer. During monitoring, the plurality of biomarkers can be analyzed across one or more timepoints. In various embodiments, a subject of the patient population is monitored for the plurality of biomarkers once every week, once every month, once every 2 months, once every 3 months, once every 4 months, once every 5 months, once every 6 months, once every 7 months, once every 8 months, once every 9 months, once every 10 months, once every 11 months, once every year, once every 18 months, once every 2 years, once every 3 years, once every 4 years, once every 5 years, or once every 10 years.

In particular embodiments, a subject is determined to be eligible for the multi-tier process by testing for the plurality of biomarkers across the one or more timepoints. For example, if a change in the levels of the plurality of biomarkers for a subject satisfies a threshold value, the subject is determined to be eligible for the multi-tier process, monitoring can cease for the subject and the subject undergoes the multi-tier process, as is described in further detail herein.

### Flow Diagram for Improving Precision of a Cancer Test

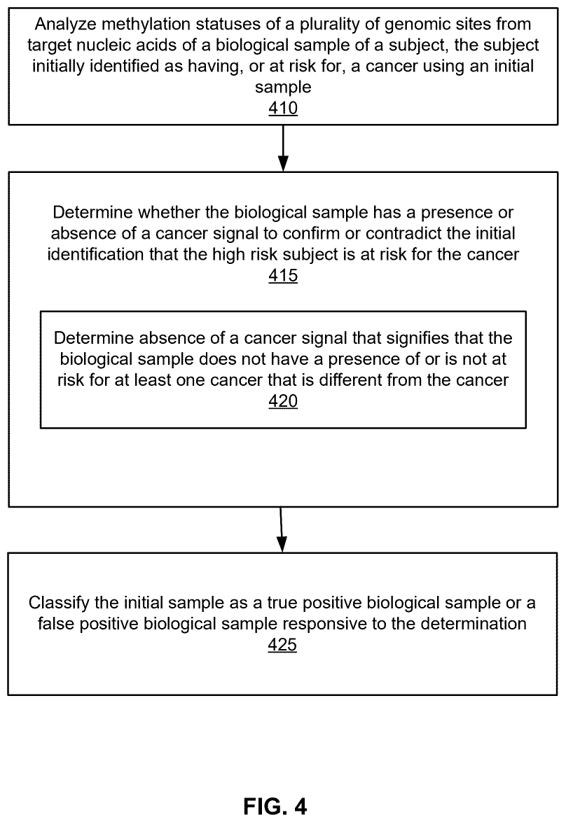

FIG. 4 shows an example flow diagram for improving precision of a cancer test, in accordance with a first embodiment. In particular embodiments, the flow diagram shown in FIG. 4 is useful for improving precision of a cancer test, e.g., a GI cancer test.

Step 410 involves analyzing methylation statuses of a plurality of genomic sites from target nucleic acids of a biological sample of a subject, the subject initially identified as having, or at risk for, cancer using an initial sample. In various embodiments, the subject was initially identified as having, or at risk for, cancer by analyzing the initial sample using a cancer diagnostic test. In various embodiments, the cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample. In various embodiments, the cancer diagnostic test is a commercially available cancer diagnostic test. Examples of a cancer diagnostic test include any of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

Step 415 involves determining whether the biological sample has a presence or absence of a cancer signal to confirm or contradict the initial identification that the high risk subject is at risk for cancer. For example, the methylation statuses of the plurality of genomic sites can be indicative of whether the biological sample has a presence or absence of a cancer signal. In various embodiments, the plurality of genomic sites comprise a portion of CpGs within a CGI, such as a portion of CpGs within at least a portion of one or more CGIs in Tables 1-4.

As shown in FIG. 4, step 415 may, in various embodiments, include the substep of step 420. For example, step 420 involves determining absence of a cancer signal that signifies that the biological sample does not have a presence of or is not at risk for at least one cancer that is different from the cancer. In particular embodiments, step 420 involves determining absence of a cancer signal that signifies that the biological sample does not have a presence of or is not at risk for a different cancer. In various embodiments, the different cancer is one or more of liver or lung cancer. Thus, step 420 involves determining additional or orthogonal cancer information in relation to the prior analysis of the initial sample using the cancer diagnostic test.

Step 425 involves classifying the initial sample as a true positive biological sample or a false positive biological sample responsive to the determination. In particular embodiments, the classification of the initial sample detects false positive samples at an accuracy of at least 80% sensitivity at 90% specificity.

### Cancers

Methods disclosed herein are useful for detecting a true positive or a false positive initial sample result in a subject. The subject may have been initially identified as having, or at risk for, a cancer. In various embodiments, the subject may be suspected of having a cancer, but may not have been previously diagnosed with a cancer. In various embodiments, the subject is healthy and is not yet suspected of having a cancer. In various embodiments, the subject may have been previously diagnosed with a cancer and receives an intervention for treating the cancer. For example, the subject may have previously received a tumor therapeutic for treating the cancer. In certain embodiments, a cancer is an early-stage health cancer, e.g., prior to development of symptoms.

In various embodiments, the cancer is an early stage cancer. In various embodiments, the cancer is a preclinical phase cancer. In various embodiments, the cancer is a stage I cancer. In various embodiments, the cancer is a stage II cancer.

In various embodiments, the cancer is any of an acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

In particular embodiments, methods disclosed herein are useful for detecting a true positive or a false positive initial sample result in a subject initially identified as having, or at risk for, a gastrointestinal (GI) cancer. Examples of GI cancer include esophageal cancer, gastric cancer, colorectal cancer, and anal cancer. In various embodiments, methods disclosed herein are useful for determining additional information that is orthogonal to the information from the previous GI cancer diagnostic test used to analyze the initial sample. For example, additional information can include presence or absence of one or more non-GI cancers. Examples of non-GI cancer include any of acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gastrointestinal cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In particular embodiments, the non-GI cancer is liver cancer or lung cancer. In particular embodiments, the non-GI cancer is liver cancer. In particular embodiments, the non-GI cancer is lung cancer.

### Computer Implementation

The methods of the invention, including the methods of detecting a true positive or a false positive initial sample result in a subject initially identified as having, or at risk for, gastrointestinal (GI) cancer, are, in some embodiments, performed on one or more computers. In particular embodiments, the step of analyzing methylation statuses of nucleic acids (e.g., step 125 shown in FIG. 1), or substeps thereof, is performed on one or more computers.

In various embodiments, the methods of detecting a true positive or a false positive initial sample result in a subject initially identified as having, or at risk for, gastrointestinal (GI) cancer can be implemented in hardware or software, or a combination of both. In one embodiment, a machine-readable storage medium is provided, the medium comprising a data storage material encoded with machine readable data which, when using a machine programmed with instructions for using said data, is capable of displaying data (e.g., methylation data) and results of analysis. Such data can be used for a variety of purposes, such as identifying true positive samples and/or identifying false positive samples. The disclosed methods can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods disclosed herein, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

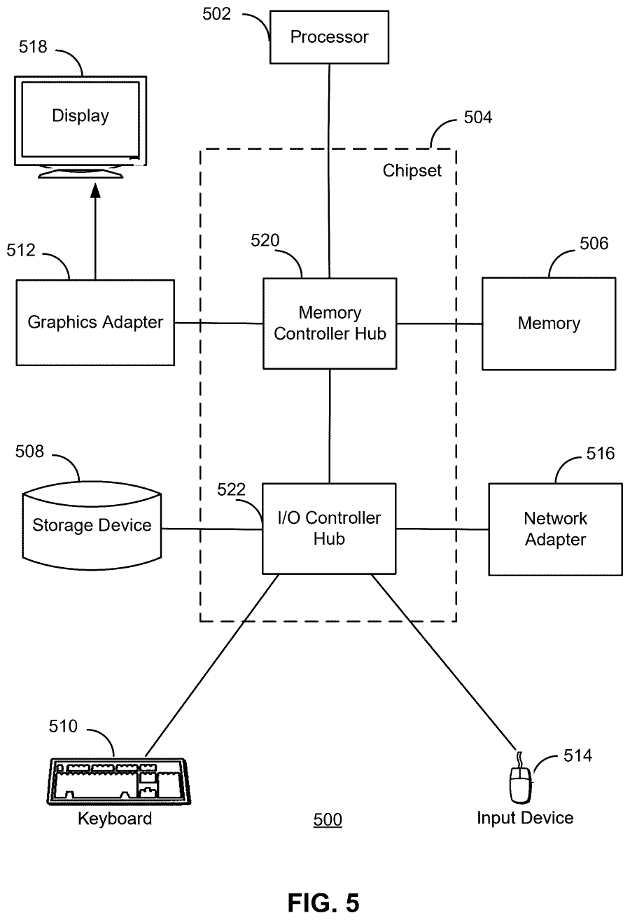

FIG. 5 illustrates an example computer for implementing the entities shown in FIGS. 1, 2A-2B, and 3-4. The computer 500 includes at least one processor 502 coupled to a chipset 504. The chipset 504 includes a memory controller hub 520 and an input/output (I/O) controller hub 422. A memory 506 and a graphics adapter 512 are coupled to the memory controller hub 520, and a display 518 is coupled to the graphics adapter 512. A storage device 508, an input device 514, and network adapter 516 are coupled to the I/O controller hub 522. Other embodiments of the computer 500 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input device 514 is a touch-screen interface, a mouse, track ball, or some combination thereof, and is used to input data into the computer 500. The keyboard 510 may be another device for inputting data into the computer 500. In some embodiments, the computer 500 may be configured to receive input (e.g., commands) from the input device 514 via gestures from the user. The graphics adapter 512 displays images and other information on the display 518. The network adapter 516 couples the computer 500 to one or more computer networks.

The computer 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

In various embodiments, methods disclosed herein can be performed on a single computer 500 or multiple computers 500 communicating with each other through a network such as in a server farm. In various embodiments, the computers 500 can lack some of the components described above, such as graphics adapters 512, and displays 518.

1. A method for detecting a true positive or a false positive initial sample result in a subject initially identified as having, or at risk for, cancer, the method comprising:
analyzing methylation statuses of a plurality of genomic sites from target nucleic acids of a biological sample of the subject;
determining whether the biological sample has a presence or absence of a cancer signal to confirm or contradict the initial identification that the high risk subject is at risk for cancer, wherein the absence of the cancer signal signifies that the biological sample does not have a presence of or is not at risk for a plurality of cancers, wherein at least one cancer of the plurality of cancers is a different cancer that differs from the cancer for which the subject was initially identified as high risk; and
classifying the initial sample as a true positive biological sample or a false positive biological sample responsive to the determination.

2. The method of claim 1, wherein the subject initially identified as at risk of cancer is undergoing or has previously undergone a colonoscopy.

3. The method of claim 1 or 2, wherein the biological sample is obtained during a colonoscopy of the subject.

4. The method of any one of claims 1-3, wherein the cancer is a gastrointestinal (GI) cancer.

5. The method of claim 4, wherein the GI cancer is any of esophageal cancer, stomach cancer, small intestine cancer, colorectal cancer, or rectal cancer.

6. The method of any one of claims 1-5, wherein the initial sample result was obtained using an initial cancer diagnostic test.

7. The method of claim 6, wherein cancer diagnostic test analyzed an initial blood or stool sample of the subject.

8. The method of claim 7, wherein the initial blood or stool sample and the biological sample are different samples.

9. The method of any one of claims 6-8, wherein the initial cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample,

10. The method of any one of claims 6-9, wherein the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

11. The method of any one of claims 6-10, wherein determining that the sample lacks the cancer signal comprises determining cancer information that is orthogonal to the information from the previous cancer diagnostic test.

12. The method of any one of claims 1-11, wherein the different cancer is one of acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer.

13. The method of any one of claims 1-12, wherein the different cancer is one or more of liver or lung cancer.

14. The method of any one of claims 1-13, wherein the absence of the cancer signal signifies that the sample does not have or is not at risk for liver or lung cancer.

15. The method of any one of claims 1-14, wherein the plurality of genomic sites comprise a plurality of CpG sites.

16. The method of claim 15, wherein the plurality of CpG sites are located in one or more CpG islands or portions of one or more CpG islands shown in Tables 1-4.

17. The method of any one of claims 1-16, wherein the methylation statuses of the plurality of genomic sites are obtained by performing an assay, wherein the assay comprises performing one or more of:
a. sequencing of target nucleic acids;
b. hybrid capture;
c. methylation-specific PCR;
d. an assay that generates sequence information; and
e. sequencing a clone library generated from a template immortalized library.

18. The method of claim 17, wherein performing the assay comprises:
obtaining converted cell free DNA (cfDNA);
selectively amplifying target regions of the converted cfDNA; and
sequencing amplicons comprising the amplified target regions to determine the methylation statuses of the plurality of genomic sites.

19. The method of claim 18, wherein the converted cfDNA comprises bisulfite converted cfDNA.

20. The method of claim 18 or 19, wherein obtaining the converted cfDNA comprises performing bisulfite conversion of the target nucleic acids of the sample.

21. The method of any one of claims 1-20, wherein the sample from the high risk subject comprises any one of a blood sample, a stool sample, a urine sample, a mucous sample, or a saliva sample.

22. The method of claim 21, wherein the sample from the high risk subject is a blood sample.

23. The method of any one of claims 1-22, wherein analyzing methylation statuses of the plurality of genomic sites from target nucleic acids of the sample comprises performing whole genome sequencing.

24. The method of any one of claims 1-23, wherein analyzing methylation statuses of the plurality of genomic sites from target nucleic acids of the sample comprises applying a trained machine learning model.

25. The method of any one of claims 1-24, wherein the target nucleic acids comprise cell free DNA (cfDNA).

26. The method of any one of claims 1-25, wherein classifying the initial sample as a true positive sample or a false positive sample responsive to the determination comprises classifying the initial sample as a false positive responsive to determining that the sample has an absence of the cancer signal.

27. The method of claim 26, further comprising:
selecting a health regimen for the high risk subject that does not include a tumor therapeutic.

28. The method of any one of claims 1-25, wherein classifying the initial sample as a true positive sample or a false positive sample responsive to the determination comprises classifying the initial sample as a true positive responsive to determining that the sample has a presence of the cancer signal.

29. The method of claim 26, further comprising selecting a tumor therapeutic for administration to the high risk subject.

30. The method of any one of claims 1-26, wherein the method detects false positive samples at an accuracy of at least 80% sensitivity at 90% specificity.

31. A method for improving precision of a cancer test, the method comprising:
identifying a subject at risk for cancer due to an initial sample result from a cancer diagnostic test;
analyzing methylation statuses of a plurality of genomic sites from target nucleic acids of a biological sample of the subject;
determining whether the biological sample has a presence or absence of a cancer signal to confirm or contradict the initial identification that the high risk subject is at risk for cancer, wherein the absence of the cancer signal signifies that the biological sample does not have a presence of or is not at risk for a plurality of cancers, wherein at least one cancer of the plurality of cancers is a different cancer; and
classifying the initial sample as a true positive biological sample or a false positive biological sample responsive to the determination.

32. The method of claim 31, wherein the subject is undergoing or has previously undergone a colonoscopy.

33. The method of claim 31 or 32, wherein the biological sample is obtained during a colonoscopy of the subject.

34. The method of any one of claims 31-33, wherein the cancer is a gastrointestinal (GI) cancer.

35. The method of claim 34, wherein the cancer is any of esophageal cancer, stomach cancer, small intestine cancer, colorectal cancer, or rectal cancer.

36. The method of any one of claims 31-35, where the cancer diagnostic test analyzed an initial blood or stool sample of the subject.

37. The method of claim 36, wherein the initial blood or stool sample and the biological sample are different samples.

38. The method of any one of claims 31-37, wherein the initial cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample,

39. The method of any one of claims 31-38, wherein the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

40. The method of any one of claims 31-39, wherein determining that the sample lacks the cancer signal comprises determining cancer information that is orthogonal to the information from the previous cancer diagnostic test.

41. The method of any one of claims 31-40, wherein the different cancer is one of acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer.

42. The method of any one of claims 31-41, wherein the different cancer is one or more of liver or lung cancer.

43. The method of any one of claims 31-42, wherein the absence of the cancer signal signifies that the sample does not have or is not at risk for liver or lung cancer.

44. The method of any one of claims 31-43, wherein the plurality of genomic sites comprise a plurality of CpG sites.

45. The method of claim 44, wherein the plurality of CpG sites are located in one or more CpG islands or portions of one or more CpG islands shown in Tables 1-4.

46. The method of any one of claims 31-45, wherein the methylation statuses of the plurality of genomic sites are obtained by performing an assay, wherein the assay comprises performing one or more of:
a. sequencing of target nucleic acids;
b. hybrid capture;
c. methylation-specific PCR;
d. an assay that generates sequence information; and
e. sequencing a clone library generated from a template immortalized library.

47. The method of claim 46, wherein performing the assay comprises:
obtaining converted cell free DNA (cfDNA);
selectively amplifying target regions of the converted cfDNA; and
sequencing amplicons comprising the amplified target regions to determine the methylation statuses of the plurality of genomic sites.

48. The method of claim 47, wherein the converted cfDNA comprises bisulfite converted cfDNA.

49. The method of claim 47 or 48, wherein obtaining the converted cfDNA comprises performing bisulfite conversion of the target nucleic acids of the sample.

50. The method of any one of claims 31-49, wherein the sample from the high risk subject comprises any one of a blood sample, a stool sample, a urine sample, a mucous sample, or a saliva sample.

51. The method of claim 50, wherein the sample from the high risk subject is a blood sample.

52. The method of any one of claims 31-51, wherein analyzing methylation statuses of the plurality of genomic sites from target nucleic acids of the sample comprises performing whole genome sequencing.

53. The method of any one of claims 31-52, wherein analyzing methylation statuses of the plurality of genomic sites from target nucleic acids of the sample comprises applying a trained machine learning model.

54. The method of any one of claims 31-53, wherein the target nucleic acids comprise cell free DNA (cfDNA).

55. The method of any one of claims 31-54, wherein classifying the initial sample as a true positive sample or a false positive sample responsive to the determination comprises classifying the initial sample as a false positive responsive to determining that the sample has an absence of the cancer signal.

56. The method of claim 55, further comprising:
selecting a health regimen for the high risk subject that does not include a tumor therapeutic.

57. The method of any one of claims 31-56, wherein classifying the initial sample as a true positive sample or a false positive sample responsive to the determination comprises classifying the initial sample as a true positive responsive to determining that the sample has a presence of the cancer signal.

58. The method of claim 57, further comprising selecting a tumor therapeutic for administration to the high risk subject.

59. The method of any one of claims 31-58, wherein the method detects false positive samples at an accuracy of at least 80% sensitivity at 90% specificity.

60. The method of any one of claims 1-59, wherein prior to the subject having been initially identified as having, or at risk for, cancer, monitoring the subject over one or more timepoints.

61. The method of claim 60, wherein monitoring the subject over one or more timepoints comprises determining levels of a plurality of biomarkers across the one or more timepoints.

62. A method for improving precision of one or more cancer tests, the method comprising:
for each of one or more previously screened subjects of a patient population;
analyzing methylation statuses of a plurality of genomic sites from target nucleic acids of a biological sample of the previously screened subject;
determining whether the biological sample of the previously screened subject has a presence or absence of a cancer signal to confirm or contradict an initial identification using an initial sample obtained from the previously screened subject, wherein the absence of the cancer signal signifies that the biological sample does not have a presence of or is not at risk for a plurality of cancers; and
classifying the initial sample as a true positive biological sample or a false positive biological sample responsive to the determination.

63. The method of claim 62, wherein at least one cancer of the plurality of cancers is a different cancer that differs from the cancer for which the previously screened subject was initially identified as high risk.

64. The method of claim 62 or 63, wherein the method achieves at least a 60% positive predictive value.

65. The method of any one of claims 62-64, wherein the method achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value.

66. The method of any one of claims 62-65, wherein each of the one or more previously screened subjects is a symptomatic patient exhibiting one or more symptoms of a cancer.

67. The method of any one of claims 62-66, wherein the initial identification using the initial sample obtained from the previously screened subject was determined using a cancer diagnostic test that analyzed an initial blood or stool sample of the previously screened subject.

68. The method of claim 67, wherein the initial blood or stool sample and the biological sample are different samples.

69. The method of any one of claim 67 or 68, wherein the cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample,

70. The method of any one of claim 67 or 68, wherein the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

71. The method of any one of claims 62-70, wherein prior to the previously screened subject having been initially identified as having, or at risk for, cancer, monitoring the subject over one or more timepoints.

72. The method of claim 71, wherein monitoring the subject over one or more timepoints comprises determining levels of a plurality of biomarkers across the one or more timepoints.

73. A method for improving precision of a cancer test for detecting cancer in subjects of a patient population, the method comprising:
monitoring subjects of the patient population by screening the subjects using initial samples obtained from the subjects, wherein the screening comprises performing an initial cancer diagnostic test;
identifying one or more subjects at risk for cancer based on results from the initial cancer diagnostic test;
for each of the one or more subjects at risk for cancer:
analyzing methylation statuses of a plurality of genomic sites from target nucleic acids of a biological sample of the subject;
determining whether the biological sample has a presence or absence of a cancer signal to confirm or contradict the initial identification that the subject is at risk for cancer based on results from the initial cancer diagnostic test, wherein the absence of the cancer signal signifies that the biological sample does not have a presence of or is not at risk for a plurality of cancers; and
classifying the results from the initial cancer diagnostic test as a true positive result or a false positive result responsive to the determination.

74. The method of claim 73, wherein at least one cancer of the plurality of cancers is a different cancer that differs from the cancer for which the subject was initially identified as at risk based on results from the initial cancer diagnostic test.

75. The method of claim 73 or 74, wherein the method achieves at least a 60% positive predictive value.

76. The method of any one of claims 73-75, wherein the method achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value.

77. The method of any one of claims 73-76, wherein at least one of the one or more subjects at risk for cancer is a symptomatic patient exhibiting one or more symptoms of a cancer.

78. The method of any one of claims 73-77, wherein the initial cancer diagnostic test analyzed an initial blood or stool sample of the subject.

79. The method of claim 78, wherein the initial blood or stool sample and the biological sample are different samples.

80. The method of claim 78 or 79, wherein the initial cancer diagnostic test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample,

81. The method of claim 78 or 79, wherein the cancer diagnostic test comprises one or more of COLOGUARD® (Exact Sciences), SHIELD™ (Guardant Health), Freenome Multiomic Blood test (Freenome), Everlywell fecal immunohistochemical test (FIT), Pinnacle Biolabsl FIT, iDNA HPV test, and imaware Prostate Cancer screening test.

82. The method of any one of claims 62-70, wherein monitoring subjects of the patient population comprises monitoring the subjects over one or more timepoints.

83. The method of claim 82, wherein monitoring the subjects over one or more timepoints comprises determining levels of a plurality of biomarkers across the one or more timepoints.
